(19) United States
(12) Patent Application Publication
(10) Pub. No.: US 2012/0282232 A1
(43) Pub. Date: Nov. 8, 2012

(54) Nutrient Hydration Bar

(76) Inventors: John George Tobin, Mays Landing, NJ (US); Carole Lynn Tobin, Mays Landing, NJ (US)

(21) Appl. No.: 13/461,256

(22) Filed: May 1, 2012

Related U.S. Application Data
(60) Provisional application No. 61/481,792, filed on May 3, 2011.

(51) Int. Cl. A61K 35/74; A61P 39/06; A61P 21/00; A61K 35/66

(52) U.S. Cl. 424/93.45; 424/93.4; 424/93.5

(57) ABSTRACT

The method of the present invention comprises the administration of a complete matrix of natural whole food vitamins, minerals including necessary electrolytes, super anti-oxidants, proteins, amino acids, complex carbohydrates, fiber, lipids and bio-flavonoids with superior absorption, utilization and retention. The supplement provides a consumer or athlete enhanced hydration retention and energy which is extremely effective in enhancing exercise and sports performance.

### FIELD OF THE INVENTION

The present invention relates generally to nutritional sports supplements and methods for their administration to provide athletes and other active individuals with an easy way to maintain a hydrated body system when working at high performance levels. More specifically, the present invention relates to methods for continually hydrating the human body through the ingestion of a solid oral comestible for the quick recovery from exercise and muscle fatigue.

### BACKGROUND OF THE INVENTION

Food products such as those marketed as energy bars are gaining in popularity among all consumers. Energy bars are designed to provide a healthful nutritious serving that is high in protein, vitamins and minerals, low in fat and shaped into a bar or other convenient form. The thought of eating a healthful nutritious energy bar that is shelf stable and packaged in a portable format is appealing to most people, especially individuals who want and/or need a functional benefit from the nutrients offered by such products.

Energy bars generally fall in one of two categories: grain-based or chewy. The grain-based energy bars are primarily made of a particulate matrix held together by a binder. The chewy variety is typically comprised of ingredients that have been processed into a homogeneous mass. In either case, the matrix or mass is then pressed, extruded or molded to form bar shaped pieces that are dried or solidified. Both deliver high levels of protein, vitamins and minerals, in a low fat bar.

Many of the currently marketed products do not appeal to consumers, who generally find them to be dry and tasteless with poor mouth feel. Moreover, the homogeneity of the chewy bar/extruded mass results in the energy bar product that usually has a singular taste throughout the homogeneous product. This leads to mediocre tasting products since nutritious ingredients such as protein, vitamins, and minerals, which often taste bad, are haphazardly mixed in with other ingredients. The combination degrades the overall taste of the product, leaving many of the presently marketed energy bars with a chalky, dry mouth feel and taste. For example, energy bars are typically fortified with protein powders, which appeals to most consumers who perceive protein as a desirable nutrient. Protein powders are typically made with whey proteins, soy proteins, egg proteins, caseins, and the like. In general, the protein powders are combined along with the other ingredients of the bar and the inclusion of protein powders tends to produce a mouth drying sensation that many consumers find undesirable. The mouth drying sensation can be so intense that some consumers will only eat an energy bar with a beverage.

The energy bars that are currently commercially available may provide some of the healthful nutritional benefits mentioned above, but they do not deliver the taste attributes desired by many consumers. As a result, some consumers settle on poor taste in order to get the healthful nutritional benefits, while many other consumers choose not to buy energy bars at all. What is missing from the marketplace and what food manufacturers have not been able to deliver, is a truly good tasting energy bar that delivers healthful nutritional benefits, i.e. high protein, fortified with vitamins and minerals, and low in fat.

There are many types of nutrition bars and other “snack” bars available on the market, and many consumers use such products as a convenient food source. For example, grain based bars such as granola bars are easy to carry and provide a healthy, good tasting food that is consumed by active people such as hikers and athletes, and other active individuals. Because grain-based nutrition bars are convenient and healthy, they have become a very popular product. One type of bar that has become popular in the recent years is commonly called an energy or “performance bar.” These products are typically formulated for use by active individuals such as athletes, and include ingredients that are intended to boost athletic performance, endurance, etc. Such energy bars provide an easy way for athletes to consume foods that are specifically formulated to improve and enhance performance of the individual. Similarly, many athletes use amino acid supplements as dietary supplements or as directed by health professionals. Athletes often consume amino acid supplements, which typically are provided in the form of capsules or tablets in order to obtain improved results in sporting events. As athletes seek to gain an edge over their competition, they frequently reach for nutritional supplements to improve their performance. A recent survey in young German elite athletes showed that over 80% of them took some version of a supplement, with most being minerals, vitamins, sport drinks, energy drinks, and carbohydrates. For this group, health promotion and performance enhancement were the most frequently stated reason for their use.

Supplements are frequently chosen to correct nutritional limitations of performance. With sweat losses amounting up to 3 liters per hour (L/h) during intense activities in extreme environments, persistent dehydration remains a concern. When the exercise duration exceeds 60 minutes, intake of a carbohydrate/electrolyte solution is necessary in order to maintain normal glucose levels and replace the electrolytes lost through sweat in order to prevent a decline in performance.

The positive effects of nutritional products can only be gained if athletes are able to consume them regularly. This is especially applicable during exercise because decreased blood flow to the digestive system results in an increased likelihood for gastrointestinal distress or nausea. The method of hydration through the administration of the supplement bar of the present invention is specifically designed to minimize potential distress as it is gluten, wheat, dairy, corn, and nut free.

U.S. Pat. No. 7,531,192 to Farber et. al. discloses oral delivery systems for functional ingredients, such as drugs, nutritional supplements, botanicals, and vitamins. The delivery systems comprise an ingestible matrix within which the functional ingredient(s) are substantially uniformly and completely dispersed and in which degradation of the functional ingredient(s) is minimized. The matrix comprises 1) one or more carbohydrates; 2) one or more sugars, sugar syrup and/or sugar alcohol; 3) one or more hydrocolloid; 4) one or more polyhydric alcohol; 5) one or more source of mono- or divalent cations, and 5) water. The combination of carbohydrate and hydrocolloid in the matrix ensures that the delivery system readily retains the solvent component and thereby prevents separation of the solvent from other components of the matrix. The invention also provides methods of preparing and using the delivery systems.

U.S. Pat. No. 7,767,248 to Overly et. al. discloses a chewy confectionary product having a high fiber content, comprising dietary fiber, water, and sweetener selected from the group consisting of corn syrup, a sweetener other than from corn syrup, sugar alcohols and mixtures thereof, corn syrup to provide a fibrous/sweetened aqueous slurry having from 60 to 74 wt. % solids, and from 25 to 40 wt. % fiber to provide a fibrous/sweetened slurry; The fibrous/sweetened slurry is hydrated during preparation to remove from 10 to 20 wt. % of the water from the fibrous/sweetened slurry which provides a cooked slurry having at least about 78 wt. % solids. Additional fiber, solids and at least about 3 wt. % of one or more humectants is added to the slurry at a temperature of at least about 100° C. to provide a chewy confectionery product having at least 42 wt. % fiber and at least 88 wt. % solids.

U.S. Pat. No. 7,220,442 to Gautam et. al. discloses a nutrition bar which incorporates protein in the form of nuggets having high levels of selected proteins. The nutrition bars are formulated to have elevated levels of protein, yet are asserted to have good taste and other organoleptic properties. The nutrition bars comprise greater than 50 wt. % of a non-soy protein such as whey or milk protein, rice protein and pea protein, especially between 51 wt. % and 99 wt. %, more preferably between 52 wt. % and 95 wt. %, most preferably 55 wt. % or above. The milk protein is preferably whey protein. The nutrition bars are preferably made using an extrusion process wherein the extrusion temperature is moderated so as to avoid damage to the whey proteins and concomitant off-taste.

U.S. Pat. No. 7,247,336 to Fitzjarrell et al teaches and claims a nutrition bar consisting of from between about 92.50 to about 97.50% of a fruit base blend comprising dehydrated fruit in an amount of at least about 60% of the blend, and grain in an amount of at least about 15% of the blend; between about 2.50 to about 7.50% free form amino acids in an amino acid blend, wherein said amino acid blend consists of histidine, leucine, phenylalanine, lysine, arginine, valine. Each amino acid is present in an amount of between 7-15% of said blend, and isoleucine, alanine, glutamine, α-ketoglutaric acid, methionine and threonine are each present in an amount between 2.0% and 10% of said blend Also included therein is a binder in an amount sufficient to form the fruit base blend and amino acid blend into a bar.

However, none of the compositions or methods known in the prior art afford the consumer or athlete a tasteful means to orally ingest a shelf stable, dry comestible composition that provides enhanced hydration retention and an energy source which is also extremely effective in improving exercise performance after ingestion.

### BRIEF DESCRIPTION OF THE DRAWINGS

FIG. 1 is a the bar graph that illustrates the average rating of perceived exertion as recorded by two groups of athletes during a workout; one group having ingested the hydration bar of the present invention, the other group not having had the bar.

FIG. 2 is a bar graph that illustrates the average amount of water consumed by the subjects of the two groups studied in FIG. 1 during training and exercises by those subjects who first ingested the hydration bar of the present invention as compared to those who did not.

FIG. 3 is a bar graph that shows the average individuals power output in watts during the exercise/physical labor tests comparing those subjects who first ingested the hydration bar of the present invention with those who did not.

FIG. 4 is a graphic portrayal that compares the average heart rate in terms of beats per minute during exercise/physical exertion of the athletes by those subjects who first ingested the hydration bar of the present invention as compared to those who did not.

FIG. 5 is a general summary of the data reported in the exertion studies.

### SUMMARY OF THE INVENTION

The method of the present invention comprises the administration of a complete matrix of natural whole food vitamins, minerals including necessary electrolytes, super anti-oxidants, proteins, amino acids, complex carbohydrates, fiber, lipids and bio-flavonoids with superior absorption, utilization and retention. Also included therein is a specific blend of one or more active ingredients derived from a bacterial or fungal fermentation broth that further enhances the supplement as a pro-biotic component. The supplement provides a consumer or athlete enhanced hydration retention and energy which is extremely effective in enhancing exercise performance.

### DETAILED DESCRIPTION OF THE INVENTION

The method of the present invention utilizes a complete matrix of natural whole food vitamins, minerals including necessary electrolytes, super anti-oxidants, proteins, amino acids, complex carbohydrates, fiber, lipids, bio-flavonoids and lipophosphates that are combined to provide an easily ingestible oral composition with superior nutrient absorption, utilization and retention. The method utilizing the composition provides hydration retention and energy enhancement with B complex vitamins and other key ingredients working synergistically. Athletes that practice this method experience quick recovery from exercise and muscle fatigue. A specific type and level of quercetin that is incorporated into the mixture is beneficial in addressing mental focus and reduced fatigue. Quercetin is a powerful antioxidant, a natural anti-histamine, and anti-inflammatory which also may help to prevent cancer, especially prostate cancer. The composition also includes an immune booster utilizing super anti-oxidants, including glutathione and vitamins C and E. The bodies own natural electrolytes are balanced through a specific mixture of nutrients and natural sea salt.

Also included therein is a specific blend of one or more active ingredients and neutraceuticals derived from a bacterial or fungal fermentation broth that further enhances the supplement as a pro-biotic component. These bacterial or fungal-derived compounds may be derived from niacin and certain strains of Lactobacillus bulgarius, L. acidophilus L. caucasicus, L. casei L. rhamnosus, Bifidobacterium animalius, and any number of the Saccharomyces cerevisiae, S. boulardii and other Saccharomyces. species.

The methods of the present invention reduce or abate the usual body requirements for water in high stress conditions by promoting hydration and water retention. This is achieved by the administration of a unique combination of whole food ingredients and nutrients that work together synergistically with pomegranate, green tea, natural sea salt, a specific ratio of amino acids and quercetin which enhances the bodies own hydration retention and natural balance of electrolytes for increased energy enhancement. This enables the athlete, after strenuous physical exertion, to realize a quick recovery from exercise/muscle fatigue while at the same time serving as an immune booster and providing enhanced mental focus.

The following examples are provided to more specifically set forth and define the process of the present invention. It is recognized that changes may be made to the specific parameters and ranges disclosed herein and that there may be a number of different ways known in the art to change the disclosed variables. And whereas it is understood that only the preferred embodiments of these elements are disclosed herein as set forth in the specification and drawings, the invention should not be so limited and should be construed in terms of the spirit and scope of the claims that follow.

### Example I

An orally ingestible hydration bar useful in the method of the present invention was prepared from the following ingredients in the amounts set forth below.

| | Weight | Percent |
|---|---|---|
| 1. Honey | 5.797 g | 18.123 |
| 2. Non GMO Soy Protein Isolate (Combined | 5.034 g | 15.732 |
| both SPI below) | | |
| Non GMO Soy Protein Isolate | 3.042 g | 9.511 |
| Non GMO Soy Protein Isolate | 1.990 g | 6.221 |
| 3. Glycerin | 3.460 g | 10.819 |
| 4. Dried Wild Blueberries | 3.009 g | 9.406 |
| 5. Cranberry Fruit Pieces | 2.999 g | 9.375 |
| 6. Cocoa Butter | 2.889 g | 9.031 |
| 7. Date Paste | 2.408 g | 7.529 |
| 8. Nutrafood Nutrients ™ | 2.114 g | 6.609 |
| 9. Alkalized Cocoa Powder | 1.528 g | 4.777 |
| 10. Palm Oil | 1.014 g | 3.170 |
| 11. Natural Flavor (Combined Natural Flavors | 0.995 g | 3.111 |
| below) | | |
| Natural Flavor - Raspberry Cream | 0.700 g | 2.188 |
| Natural Flavor - Vanilla | 0.295 g | 0.923 |
| 12. Quercetin | 0.250 g | 0.781 |
| 13. Sea Salt | 0.192 g | 0.551 |
| 14. Creatine Monohydrate | 0.115 g | 0.360 |
| 15. Green Tea | 0.050 g | 0.156 |
| 16. Pomegranate Extract | 0.050 g | 0.156 |
| 17. L-Arginine | 0.050 g | 0.156 |
| 18. L-Glutamine | 0.050 g | 0.156 |

### Example II

The method of the present invention was examined using the hydration bar of the present invention to determine if the hydration bar was well tolerated, accepted, and effective in enhancing exercise performance. The study was conducted at Wake Forest University N.C. and the Olympic Training Center (OTC) in Colorado Springs, Co. In the study, five (5) triathletes, six (6) male and three (3) females who ranged in age from 19-31 participated.

There were two phases of the study. The first phase lasted for two weeks during which the participants consumed one (1) hydration bar of the present invention per day for the first week and two (2) hydration bars for the second week. Participants completed questionnaires utilizing Likert scales and open-ended questions about the taste, tolerance, and acceptability of the product every day during the trials. The athletes consumed three bars per day during the first week. During the second week, they participated in two (2) 2-hour exercise sessions at an intensity of 60-75% of their predicted heart rate reserve. Participants completed questionnaires similar to those in Phase 1 after their exercise session. In addition, work level, water consumption, and perceived exertion were measured.

The questionnaire presented to the subjects' inquired as to their thoughts on the hydration bars' palatability and acceptance and contained twelve 9-point Likert scale questions along with 3 open-ended questions. The mean response was calculated for each question. An example of the questions is shown below:

The product had a pleasant taste.

| Strongly Agree | Agree | | | Neutral | Disagree | | | Strongly Disagree |
|---|---|---|---|---|---|---|---|---|
| 1 | 2 | 3 | 4 | 5 | 6 | 7 | 8 | 9 |

Each athlete's power output and rate of perceived exertion were recorded during bicycle rides for athletes at the OTC during phase 2. Watts were measured through the use of a compu-trainer, which displayed wattage output in real time as a measure of the degree of energy consumed and calories metabolized. The watts generated and an athletes' rating of perceived exertion (RPE) were recorded every ten minutes. Heart rate was also provided in real time with the use of a polar heart rate monitor. These were recorded every ten minutes.

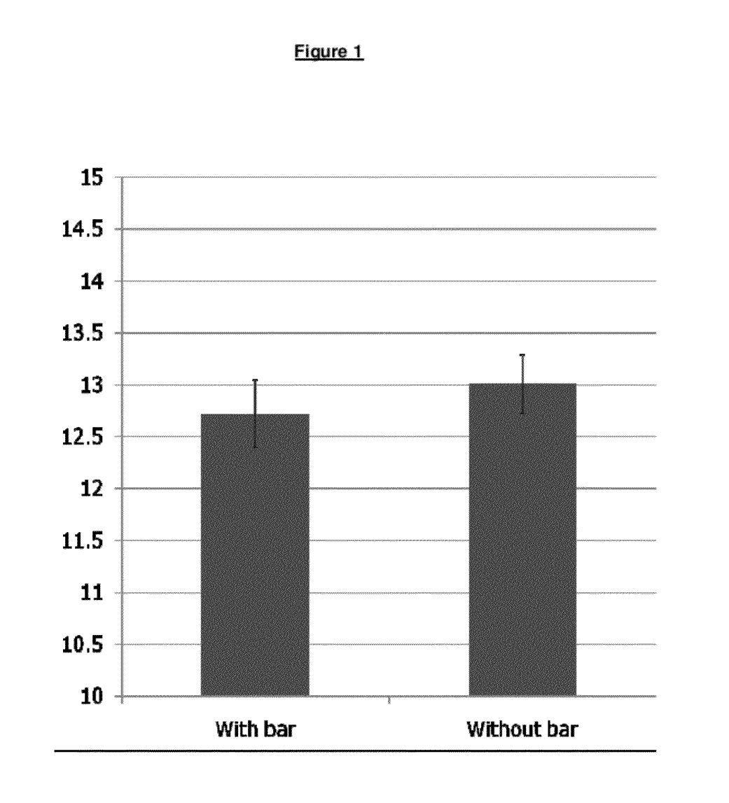

Referring now to FIG. 1, the bar graph shows the average rating of perceived exertion as recorded by the athletes during exercise, one group having ingested the hydration bar of the present invention, the other group not having had the bar. Clearly those who ingested the bar were able to carry out the assigned workload and/or workout and perceived a much lower level of physical stress (12.60) than those who did not (13.0).

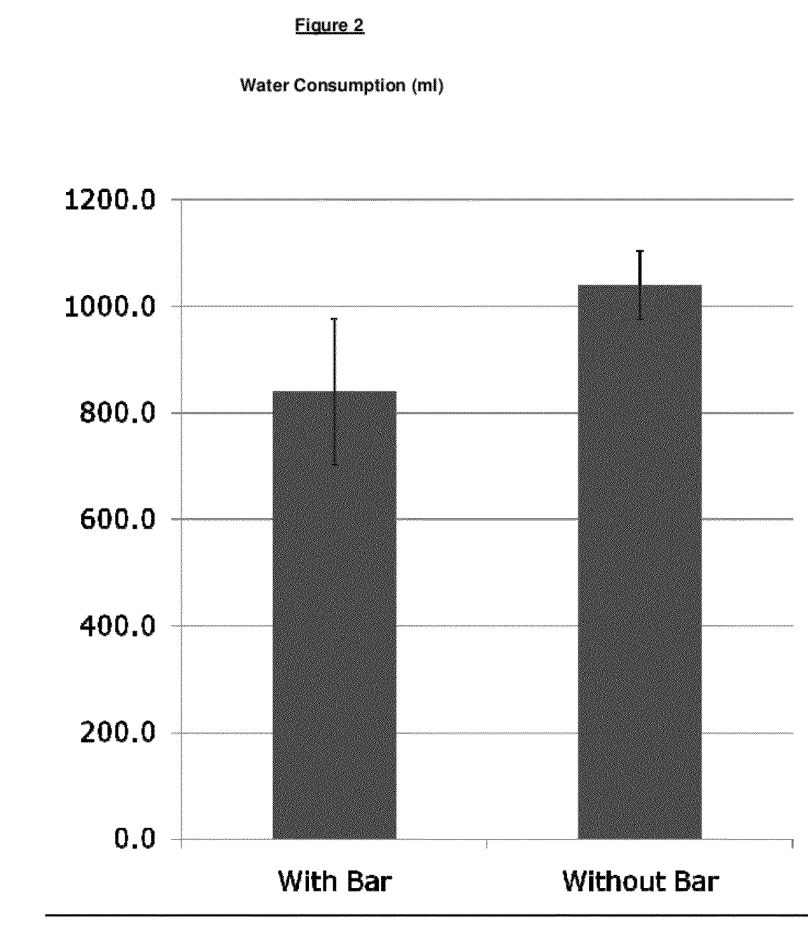

Referring now to FIG. 2, the bar graph shows the average amount of water consumed by the subjects during training and exercises by those subjects who first ingested the hydration bar of the present invention as compared to those who did not. Here again, it is clear that those who consumed the hydration bar prior to exercise/physical labor needed to consume less water (840 rating) as opposed to those who did not (1040) with a perception of a much lower level of physical stress than those who did not.

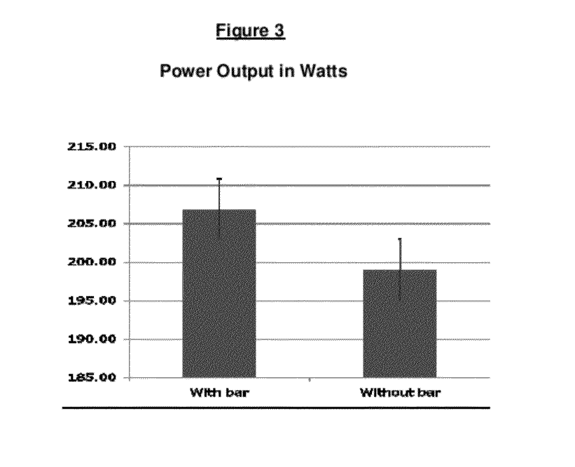

Referring now to FIG. 3, the bar graph shows the average individuals power output in watts during the exercise/physical labor tests. Those subjects who first ingested the hydration bar of the present invention as compared to those who did not had a consistently higher level of power output (206 watts average) versus those who did not.

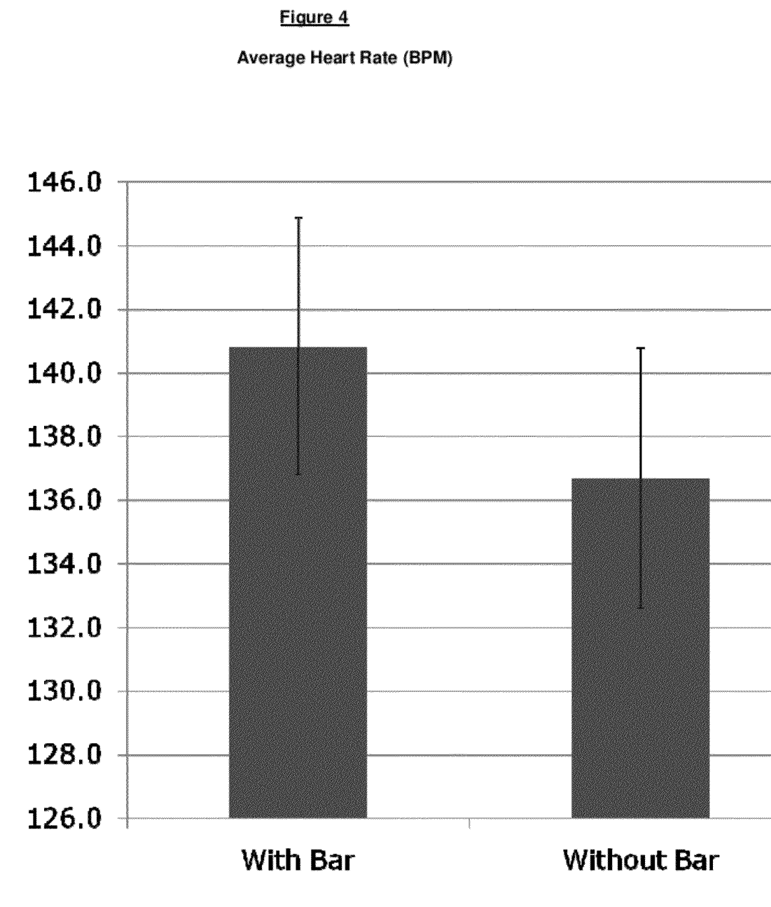

Referring now to FIG. 4, the bar graph compares the average heart rate in terms of beats per minute during exercise/physical exertion of the athletes by those subjects who first ingested the hydration bar of the present invention as compared to those who did not. Here again, it is clear that those who consumed the hydration bar prior to exercise/physical labor were able to achieve and maintain a higher average rate of about 140.8 BPM as opposed to those who did not (136.8) with a perception of a much lower level of physical stress.

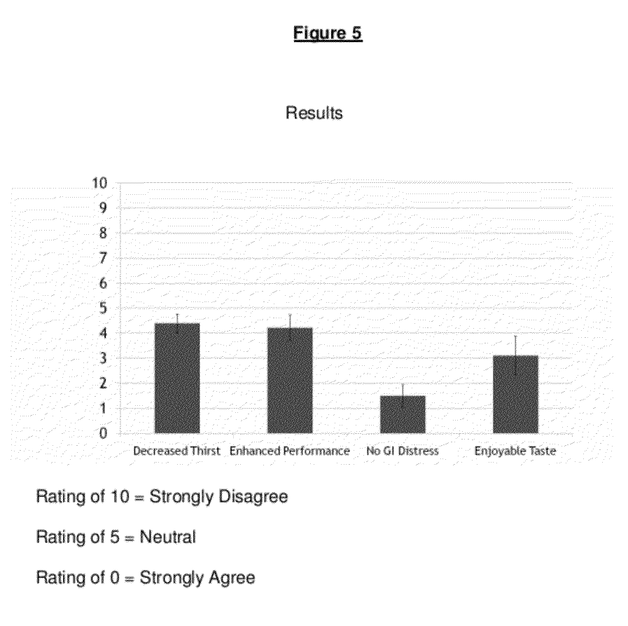

FIG. 5 generally summarizes the findings of studies and shows that it was consistently found that when athletes consumed the energy-hydration bar of the method of the present invention they felt better during exercise and were able to maintain a higher power output and higher heart rate with a lower rate of perceived exertion. Without being bound to any theory, this could be because of a glycogen deficit which results when athletes exercised without consuming the bar. The increased heart rate is likely from the athletes' ability to carry a higher work load after consuming the energy/hydration bar. Similarly, athletes consumed significantly less water during physical exertion when they used the energy hydration bars which is due to the concentrated doses of electrolytes such as sodium, potassium, and calcium in the bar. Athletes also showed that they enjoyed the taste of the bar and that there was also very limited gastro-intestinal (GI) distress.

What is claimed is:

1. A nutrient hydration bar comprising a complete matrix of natural whole food vitamins, minerals including necessary electrolytes, one or more pro-biotic compounds, super anti-oxidants, proteins, amino acids, complex carbohydrates, fiber, lipids and bio-flavonoids.

2. The hydration bar of claim 1 wherein said vitamins are selected from the group consisting of natural whole food vitamins.

3. The hydration bar of claim 2 wherein said vitamins are selected from the group consisting of beta-carotene, B complex, thiamine, riboflavin, niacin, pantothenic acid, pyridoxine, cyanocobalamin, biotin, choline, inositol, bioflavonoids, coenzyme Q, ascorbic acid, Vitamin E and Vitamin D.

4. The hydration bar of claim 3 wherein said minerals are selected from the group consisting of calcium, magnesium, iron, manganese, phosphorus, molybdenum, selenium, copper, zinc, potassium, chromium, and cesium.

5. The hydration bar of claim 4 wherein said anti-oxidants are selected from the group consisting of quercetin, creatine monohydrate, glutathione, flavonoids, lycopene, polyphenols, superoxide dismutase, beta-carotene, selenium and mixtures thereof.

6. The hydration bar of claim 5 wherein said amino acids are selected from the group consisting of L-arginine, l-glutamate, l-phenylalanine, homocysteine, aspartic acid, theronine, arginine, cysteine, lysine tryptophan and mixtures thereof.

7. The hydration bar of claim 6 wherein said probiotic blend is one or more active ingredients derived from a bacterial or fungal fermentation broth derived from niacin and certain strains of Lactobacillus bulgarius, L. acidophilus, L. caucasicus, L. reateri, L. casei, Bifidobacterium animalius, Saccharomyces cerevisiae, S. boulardi, other fungal species and mixtures thereof.

8. The hydration bar of claim 7 further comprising honey, soy protein isolate glycerin cocoa butter, palm oil, green tea, and pomegranate extract

9. The method of claim 8 wherein said hydration bar is formulated as a ready to eat solid.

10. A method for the hydration of an individual for the quick recovery from exercise and muscle fatigue undergoing physical exertion through the ingestion of a hydration bar consisting a complete matrix of natural whole food vitamins, minerals including necessary electrolytes, one or more pro-biotic compounds, super anti-oxidants, proteins, amino acids, complex carbohydrates, fiber, lipids and bio-flavonoids.

11. The method of claim 10 wherein said vitamins are selected from the group consisting of natural whole food vitamins.

12. The method of claim 11 wherein said vitamins are selected from the group consisting of beta-carotene, B complex, thiamine, riboflavin, niacin, pantothenic acid, pyridoxine, cyanocobalamin, biotin, choline, inositol, bioflavonoids, coenzyme Q, ascorbic acid, Vitamin E and Vitamin D.

13. The method of claim 12 wherein said minerals are selected from the group consisting of calcium, magnesium, iron, manganese, phosphorus, molybdenum, selenium, copper, zinc, potassium, chromium, and cesium.

14. The method of claim 13 wherein said anti-oxidants are selected from the group consisting of quercetin, creatine monohydrate, glutathione, flavonoids, lycopene, polyphenols, superoxide dismutase, beta-carotene, selenium and mixtures thereof.

15. The method of claim 14 wherein said hydration bar further comprises amino acids selected from the group consisting of L-arginine, l-glutamate, L-phenylalanine, homocysteine, aspartic acid, theronine, arginine, cysteine, lysine tryptophan and mixtures thereof.

16. The method of claim 15 wherein said probiotic blend is one or more active ingredients derived from a bacterial or fungal fermentation broth derived from niacin and certain strains of Lactobacillus bulgarius, L. acidophilus, L. caucasicus, L. reateri, L. casei, Bifidobacterium animalius, Saccharomyces cerevisiae, S. boulardi, other fungal species and mixtures thereof.

17. The method of claim 16 wherein said hydration bar further comprises honey, soy protein isolate glycerin cocoa butter, palm oil, green tea, and pomegranate extract

18. The method of claim 17 wherein said hydration bar is formulated as a ready to eat solid.

19. The method of claim 18 wherein said individual, after ingesting the hydration bar, is able to maintain a higher power output and higher heart rate with a lower rate of perceived exertion during strenuous exercise.
